(19) United States
(12) Patent Application Publication
(10) Pub. No.: US 2016/0287324 A1
(43) Pub. Date: Oct. 6, 2016

(54) METHODS AND DEVICES FOR TREATING AND PREVENTING CONDITIONS OF TUBULAR BODY STRUCTURES

(71) Applicant: The General Hosptial Corporation, Boston, MA (US)

(72) Inventors: Rahmi N. Oklu, Boston, MA (US); Martin L. Yarmush, Boston, MA (US); Alexander Goldberg, Boston, MA (US)

(21) Appl. No.: 15/038,607

(22) PCT Filed: Apr. 15, 2014
(86) PCT No.: PCT/US14/34198

§ 371 Date: May 23, 2016

Related U.S. Application Data
(60) Provisional application No. 61/907,803, filed on Nov. 22, 2013.

(51) Int. Cl. A61B 18/14; A61N 1/32

(52) U.S. Cl. CPC A61B 18/1492; A61N 1/327; A61B 2018/0041

(57) ABSTRACT

Methods and devices for treating and preventing conditions of tubular body structures, such as veins, are disclosed. In one example embodiment, there are provided methods and devices for the treatment of deep vein thrombosis. In another example embodiment, there are provided methods and devices using irreversible electroporation for sterilizing intravenous catheters.

### CROSS-REFERENCES TO RELATED APPLICATIONS

This application claims priority from U.S. Patent Application No. 61/907,803 filed Nov. 22, 2013.

### STATEMENT REGARDING FEDERALLY SPONSORED RESEARCH

Not Applicable.

### BACKGROUND OF THE INVENTION

1. Field of the Invention

The invention relates to methods and devices for treating conditions of tubular body structures, such as veins.

2. Description of the Related Art

Deep venous thrombosis (DVT) is a major public health problem associated with high morbidity and mortality. In the United States alone, approximately 900,000 people a year are diagnosed with DVT; this has not changed in the past three decades despite advancements in diagnostic imaging and medical management. Many studies have shown that the best medical management today is inadequate in the treatment of DVTs. In multiple prospective trials, the incidence of post-thrombotic syndrome (PTS) is up to 60% at 2 years despite best anticoagulation medical therapy. PTS occurs when a clot in the vein damages the valves and creates outflow obstruction. PTS significantly impacts the patient's quality of life causing persistent symptoms, such as swelling and pain, but more importantly it increases the risk of recurrence of DVT necessitating life-long anticoagulation. Extended anti-coagulation therapy is both costly and itself associated with high morbidity and mortality. There is a great-unmet need in medicine today to identify ways in which acute DVT can be effectively treated avoiding PTS.

Histology of an aging venous thrombus over time reveals a complex, dynamic process of clot organization involving cells within the walls of the vein and cells trapped within the venous thrombus. This process continues such that within 3 weeks the collagen content of the thrombus exceeds 80% rendering the thrombus resistant to thrombolysis. Anticoagulation alone only halts thrombus progression and does not influence the transformation of the clot. The thrombus is predominantly excluded from the circulation favoring clot organization to progress producing a high incidence of PTS in patients appropriately treated with best medical therapy. See, also, Oklu et al., “Detection of Extracellular Genomic DNA Scaffold in Human Thrombus: Implications for the Use of Deoxyribonuclease Enzymes in Thrombolysis”, J Vasc Intery Radiol 2012; 23:712-718.

The standard of medical practice in the treatment of venous thrombosis is anticoagulation for up to six months. If there is persistent thrombus and evidence of post-thrombotic syndrome, then anticoagulation is continued possibly life-long. The decision to treat acute DVT using percutaneous modalities as an adjunct to anticoagulation begins with the selection of the right patient population that will most benefit from this technique. This is currently limited to those patients that have a threatened limb or a break-through pulmonary embolism despite anticoagulation.

Pharmacologic thrombolysis is currently performed using recombinant tissue plasminogen activator (tPA). tPA is a serine protease that cleaves the ubiquitous inactive plasminogen to produce the active form of the protease, plasmin. Plasmin, also a serine protease, initiates the process of fibrinolysis. Because fresh thrombus is fibrin-rich and aged thrombus is connective tissue-rich, the results of fibrinolysis vary. As a result of reduced efficacy of tPA in older thrombus, practice guidelines recommend pharmacologic venous thrombolysis only within 14 days of clot formation.

Given the potential morbidity and mortality associated with pharmacologic thrombolysis therapy, the benefits of the procedure must outweigh its risks. The use of catheter directed therapy (CDT) with mechanical thrombectomy (MT) in the treatment of patients with a threatened limb, progression of DVT or worsening of symptoms despite optimal anticoagulation maybe considered appropriate if there is no contraindication to the use of tPA. There are multiple ways to perform CDT, as well as MT, and the choice of method depends on operator experience and the availability of potentially costly equipment.

The initial step in the percutaneous treatment of acute DVT patients is to define the extent of the thrombus using digital subtraction angiography. Based on the angiographic appearance, the age of the patient and the clinical history, the operator must then estimate the age of the thrombus that is likely to be the cause of the presenting symptoms. If the thrombus is within two weeks old or if there is a suspicion that there may be an acute component superimposed on an existing chronic thrombus, the operator may either choose CDT with alteplase (a recombinant tissue plasminogen activator) therapy or choose to perform CDT with MT to debulk the thrombus load and then perform CDT if needed.

Currently, there are two major types of catheters available in the market for use to perform CDT. The older, traditional type of catheter has holes on multiple sides and typically requires a drug infusion time of 24 hours prior to angiographic evaluation to assess for clot lysis. The newer generation catheters employ ultrasound technology to agitate the clot and expose greater binding sites for the thrombolytic drug to work on, while simultaneously delivering 0.01 mg/kg/hr of tPA through the same catheter. Ultrasound coupled with multi-side holed catheters has been shown to significantly decrease the time-to-thrombolysis by approximately 40% and uses 50-70% less lytic drug. If there is residual thrombus remaining following an initial session with CDT, one option is to replace and/or reposition the multi-side holed catheters and continue treatment for an additional day. Complete resolution of the DVT may not always occur following the initial session and mechanical fragmentation, maceration or aspiration of the thrombus may be necessary to further decrease the thrombus load.

There are a variety of tools in the armamentarium of an interventionalist for MT. Selection of one device over another is often case-dependent and relies on the operator's preference. The simplest form of MT is to use non-compliant, high-pressure angioplasty balloons to crush the thrombus in between the balloon and the vessel wall. Balloon angioplasty is often sufficient for most acute to subacute blood clots. If there is residual thrombus remaining following balloon angioplasty, then strategies employing mechanical devices that physically agitate the clot may be considered. For example, one such device uses compliant balloons separated by a fixed distance of sinusoidal shaped catheter containing multiple side-holes to deliver thrombolytics such as alteplase. Other devices employ a pulse-spray type of strategy where a high-pressure nozzle at the end of the catheter delivers up to 15 mg of intra-thrombus tPA followed by a 15-20 minute period to allow the drug to lyse the thrombus. During the second-pass of the catheter through the thrombus, the aspiration port is opened to allow collection of the macerated thrombus in a bag.

In one example CDT/MT method, the common femoral vein of the patient is accessed using an 18-gauge needle; the access site is secured using a 0.035 inch wire. Over this wire, a 7 French 11 cm sheath is placed and a 5 French Kumpe catheter is delivered through the occluded vein into the patent venous segment. A pull-back angiography is performed to determine the degree of the thrombus load. The Kumpe catheter is removed over a 0.035 Rosen wire; this wire remains as the working wire to push thrombolysis and MT devices through the thrombus. Once the intervention is completed, all catheters and wires including the sheath are removed and hemostasis is achieved via manual compression.

Occasionally, despite complete lysis of the acute thrombus, the chronic component is often refractory to CDT and MT treatment. Typically it is not possible to macerate the chronic thrombus using extremely high-pressure balloon angioplasty; occasionally a successful intervention of the acute thrombus may reveal an obstruction as the mechanical cause of the patient's DVT. In these situations, a stent may be placed to maintain flow within the chronically obstructed vein to prevent recurrence of DVT. Typically, it is difficult to age thrombi; catheter-directed thrombolysis is often performed only to discover that it had minimal effect, necessitating extensive angioplasty and even stent placement to restore flow.

Other venous disorders are common. For example, prevalence estimates for varicose veins are <1% to 73% in females and 2% to 56% in males (Ann Epidemiol. 2005 March; 15(3):175-84). Varicose veins can thrombose and cause thrombophlebitis.

Catheters are used in many medical procedures including in the treatment of deep venous thrombosis and varicose veins. There are also a number of catheters that are used for prolonged access to a patient's vascular system, urinary system, or other bodily conduits. Such catheters include, without limitation, central venous catheters, peripherally-inserted central catheters (also known as “PICC lines”), dialysis catheters, percutaneous nephrostomy (PCN) catheters, gastrostomy tubes, and drainage catheters, such as urinary catheters, biliary drainage catheters or catheters to drain abscesses that often develop following surgery. These catheters may be implanted into a patient for an extended period of time to allow for multiple treatments, such as the delivery of therapeutic agents, dialysis treatments or provide drainage of bodily fluids or infectious material. Long-term catheters typically stay in for more than twenty-eight days; occasionally, drainage catheters such as nephrostomy, biliary or dialysis catheters may remain life-long. Use of such catheters eliminates the need for multiple placements of single-use catheters.

Infections account for some of the complications associated with long-term catheters. For example, during an implant-associated infection, an infectious agent (e.g., fungi, micro-organisms, parasites, viral pathogens, or bacterial pathogens) interferes with the normal functioning of a patient. Even with current sterilization procedures, implant-associated infections remain a challenge with long term catheters. Implant-associated infections may be difficult to detect, problematic to cure, and at times expensive to manage. For example, in cases where the infection fails to subside quickly, it becomes necessary to remove the catheter.

Implant-associated infections can result from bacterial adhesion and subsequent biofilm formation near an implantation site. Alternatively, a previously sterile catheter may become infected from infections elsewhere in the body i.e., surgical infections, urinary tract infection and bacteremia regardless of cause. Biofilm-forming microorganisms sometimes colonize the surface of a catheter. Once a biofilm-induced infection takes hold, it can prove difficult to treat. In the case of catheters, for example, infectious agents may make their way from an insertion site into an outer surface of an indwelling portion of a catheter. Also, contamination of an outer portion, such as a venous line of catheter, may initiate migration of an infectious agent along an internal passageway.

Unfortunately, catheter infection is associated with significant morbidity and mortality, and intravenous antibiotics may not be useful as bacteria shield themselves via biofilm. Thus, there is also a need for an improved catheter with means for maintaining sterility of the catheter.

In summary, there is a need for improved methods and devices for treating conditions of tubular body structures.

### SUMMARY OF THE INVENTION

In one aspect, the invention provides a method for treating thrombosis in a subject. The method includes the step of inducing irreversible electroporation of cells of a thrombus and cells of a vessel wall in the subject. In one version of the method, a pair of electrodes are positioned adjacent a site of the thrombus or within the thrombus, and an electric potential is created between the pair of electrodes. The electric potential is sufficient to induce irreversible electroporation of cells of the thrombus and cells of the vessel wall including the endothelium of the intima layer and smooth muscle cells of the media layer. The electric potential is sufficient to induce thermal damage to vein tissue adjacent the thrombus. The electric potential can be created by a voltage pulse generator in electrical communication with the pair of electrodes.

In one form, the pair of electrodes are wound in spaced parallel relationship around a wire. The wire can have a diameter of 0.5 to 3 millimeters. The pair of electrodes can be positioned in a treatment zone having a longitudinal length that extends from a distal end of the wire. The longitudinal length can be between 1 to 10 centimeters. The pair of electrodes may span an entire length of the thrombus.

In another form, the pair of electrodes are wound in spaced parallel relationship around a catheter. The catheter can have a size of 3 to 8 on a French catheter scale. The pair of electrodes can be positioned in a treatment zone having a longitudinal length that extends from a distal end of the catheter. The longitudinal length can be between 1 to 10 centimeters. The pair of electrodes may span an entire length of the thrombus.

The method inhibits collagen deposition within the thrombus. The thrombosis can be a venous thrombosis. The thrombosis can be a deep vein thrombosis.

The method may further include the step of administering an anticoagulant to the subject.

The thrombus may be less than two weeks old, or any age. The thrombus can have less than 80% collagen, preferably less than 70% collagen, preferably less than 60% collagen, preferably less than 50% collagen, preferably less than 40% collagen, preferably less than 30% collagen, preferably less than 20% collagen, and preferably less than 10% collagen.

In another aspect, the invention provides an imaging method including the steps of positioning a pair of electrodes adjacent a site of a thrombus or within a thrombus in a subject; creating an electric potential between the pair of electrodes, wherein the electric potential is sufficient to induce irreversible electroporation of cells of the thrombus; and acquiring an image of the site of the thrombus in the subject.

In another aspect, the invention provides a method for promoting weight loss in a subject. The method includes the step of inducing irreversible electroporation of cells lining a stomach in the subject. In one version of the method, a pair of electrodes are positioned adjacent cells lining the stomach, and an electric potential is created between the pair of electrodes, wherein the electric potential is sufficient to induce irreversible electroporation of cells lining the stomach. The electric potential can be created by a voltage pulse generator in electrical communication with the pair of electrodes. In one form, the pair of electrodes are wound in spaced parallel relationship around a wire. The wire can have a diameter of 0.5 to 3 millimeters. In another form, the pair of electrodes are wound in spaced parallel relationship around a catheter. The catheter can have a size of 3 to 8 on a

French catheter scale.

In another aspect, the invention provides a catheter system including a body having an outer wall extending from a proximal end to a distal end wherein the body defines at least one lumen; a pair of electrodes disposed in the outer wall of the body; and a voltage generator in electrical communication with the pair of electrodes.

The voltage generator creates an electric potential between the pair of electrodes when the voltage generator is activated wherein the electric potential is sufficient to induce irreversible electroporation of cells of an infectious agent in contact with or adjacent the outer wall of the body. Preferably, the electric potential is insufficient to induce thermal damage to tissue adjacent an indwelling portion of the outer wall of the body. The voltage generator can be a voltage pulse generator. The infectious agent can be microorganisms capable of growing a biofilm on the outer wall of the body. The microorganisms can be bacteria.

The catheter system can further include one or more additional pairs of electrodes disposed in the outer wall of the body, wherein each additional pair of electrodes is in electrical communication with the voltage generator. The pair of electrodes and each additional pair of electrodes can be equally spaced around the outer wall of the body.

In one form, the pair of electrodes and each additional pair of electrodes include a first surface in contact with the outer wall of the body and a second surface that is not in contact with the outer wall of the body. In another form, the pair of electrodes and each additional pair of electrodes are embedded within the outer wall of the body such that the pair of electrodes and each additional pair of electrodes are surrounded by material comprising the outer wall of the body. The pair of electrodes can be positioned in a treatment zone of outer wall of the body, wherein the treatment zone has a length that extends from a distal end of the body to a location spaced from the proximal end of the body.

The body of the catheter system can have various applications. The body can comprise a central venous catheter. The body can comprise a peripherally-inserted central catheter. The body can comprise a dialysis catheter. The body can comprise a percutaneous nephrostomy catheter. The body can comprise a gastrostomy tube. The body can comprise a urinary catheter. The body can comprise a biliary drainage catheter.

In another aspect, the invention provides a method for maintaining sterility of a catheter. The catheter includes a body having an outer wall extending from a proximal end to a distal end, wherein the body defines at least one lumen and wherein the catheter includes a pair of electrodes disposed in the outer wall of the body. An electric potential is created between the pair of electrodes, wherein the electric potential is sufficient to induce irreversible electroporation of cells of an infectious agent in contact with or adjacent the outer wall of the body. The infectious agent can be microorganisms capable of growing a biofilm on the outer wall of the body. The microorganisms can be bacteria.

A voltage generator in electrical communication with the pair of electrodes can be used to create the electric potential between the pair of electrodes. The voltage generator can be a voltage pulse generator. Preferably, the electric potential is insufficient to induce thermal damage to tissue adjacent an indwelling portion of the outer wall of the body of the catheter.

One or more additional pairs of electrodes can be disposed in the outer wall of the body of the catheter. Each additional pair of electrodes is in electrical communication with the voltage generator. The pair of electrodes and each additional pair of electrodes can be equally spaced around the outer wall of the body. The pair of electrodes and each additional pair of electrodes can include a first surface in contact with the outer wall of the body and a second surface that is not in contact with the outer wall of the body. In another form, the pair of electrodes and each additional pair of electrodes are embedded within the outer wall of the body such that the pair of electrodes and each additional pair of electrodes are surrounded by material comprising the outer wall of the body.

The step of creating an electric potential between the pair of electrodes can be repeated after a predetermined time period, such as every day or every week. The catheter can be selected from central venous catheters, peripherally-inserted central catheters, dialysis catheters, percutaneous nephrostomy catheters, gastrostomy tubes, urinary catheters, and biliary drainage catheters. The catheter may be a long term catheter.

These and other features, aspects, and advantages of the present invention will become better understood upon consideration of the following detailed description, drawings, and appended claims.

### BRIEF DESCRIPTION OF THE DRAWINGS

FIG. 1 is a side view of an electroporation system of the present disclosure that is useful in treating thrombosis in a subject.

FIG. 2 is a detailed partial perspective view of the distal end of the catheter of the electroporation system of FIG. 1 taken along line 2-2 of FIG. 1.

FIG. 3 is a detailed partial side view, similar to FIG. 2, of a distal end of another catheter of an electroporation system of the present disclosure that is useful in treating thrombosis in a subject.

FIG. 4 is a side view of a catheter system of the present disclosure that includes means for sterilizing the catheter.

FIG. 5 is a cross-sectional view of the catheter of FIG. 4 taken along line 5-5 of FIG. 4.

FIG. 6 is a cross-sectional view, similar to FIG. 5, of a distal end of another catheter of the present disclosure that includes means for sterilizing the catheter.

FIG. 7 shows in (A), a control femoral vein at day 3 wherein the black star indicates early clot organization with regions of high cellularity, and in (B), an irreversible electroporation treated femoral vein showing preservation of the venous scaffold, minimal remaining cells post-ablation, and absence of any signs of clot organization.

FIG. 8 shows: in (A) and (D), rat veins that did not receive electric field treatment; and in (B), (C) and (E), rat veins that received non-thermal, low-voltage, pulsed electric fields applied to rat deep vein thrombus. (A) to (D) shows the progression of clot organization from day 3 to day 7, respectively. (D) reveals significant clot organization where there is loss of vessel wall demarcation, extensive concentration of spindle shaped nuclei, which are fibroblasts, and near complete replacement of the thrombus matrix with connective tissue. Images (B) and (C) are treated thrombi that are 3 days old; these reveal nearly absent cells within the thrombus and vessel wall with preservation of the vessel wall, absence of any fibrin matrix attached to the vessel wall and absence of any inflammatory cells. Image (E) demonstrates a DVT that was treated 7 days prior now revealing a patent vein with minimal vessel wall disease, preservation of the vessel wall layers and absence of inflammation. These images are representative images.

FIG. 9 evidences marked organization of rat vein thrombus producing a microscopic appearance similar to human chronic thrombi. Images (A) and (B) are chronic thrombus samples that were sent to pathology for evaluation revealing near complete replacement of the matrix with connective tissue and the cellular population comprising of predominantly spindle shaped cells that are fibroblasts and rounded nuclei that are inflammatory cells.

FIG. 10 evidences decreased levels of collagen detected in rat veins that received non-thermal, low-voltage, pulsed electric fields compared to control samples. Images A and B represent an untreated 3 day DVT sample in a rat showing marked cellular infiltration, inflammation and extensive collagen-3 deposition. The treated veins, however, (images C and D) show significantly less collagen-3 deposition and no evidence of clot organization. On gross examination, the treated veins are typically easily collapsible and are often flat due to fresh blood or non-organized thrombus matrix.

FIG. 11 evidences decreased prothrombogenic extracellular traps detected in the thrombus matrix in rat veins that received non-thermal, low-voltage, pulsed electric fields compared to control samples. Images A-C demonstrate nearly absence of extracellular traps in a treated day-3 thrombus. Images D-F reveal significant extracellular traps detected by immunohistochemistry in the thrombus matrix. These images are representative images.

FIG. 12 evidences that all three of the TGF-B growth factors were significantly reduced in rat veins that received non-thermal, low-voltage, pulsed electric fields compared to control samples. Transforming growth factors are key players most notably in fibrosis; all three isoforms were reduced in the treated DVT samples. These experiments were performed using a multiplex cytokine array.

Like reference numerals will be used to refer to like parts from Figure to Figure in the following description of the drawings.

### DETAILED DESCRIPTION OF THE INVENTION

The present disclosure provides methods and devices for treating and preventing conditions of tubular body structures. In one example embodiment, there are provided methods and devices for the treatment of deep vein thrombosis. In another example embodiment, there are provided methods and devices using irreversible electroporation for sterilizing intravenous/intraperiotoneal catheters.

### I. Treatment of Deep Vein Thrombosis

Since the cells of the venous wall and the cells of the thrombus orchestrate the transformation of an acute thrombus into an organized one, it was hypothesized that ablating these cells using non-thermal irreversible electroporation (IRE) may prevent or slow the process of clot organization. This strategy can provide sufficient time to allow endogenous thrombolysis to occur while clot organization is halted or slowed. Cellular ablation of venous thrombi may potentially decrease the incidence of post-thrombotic syndrome, recurrence of deep venous thrombosis and limit the need for chronic anticoagulation therapy.

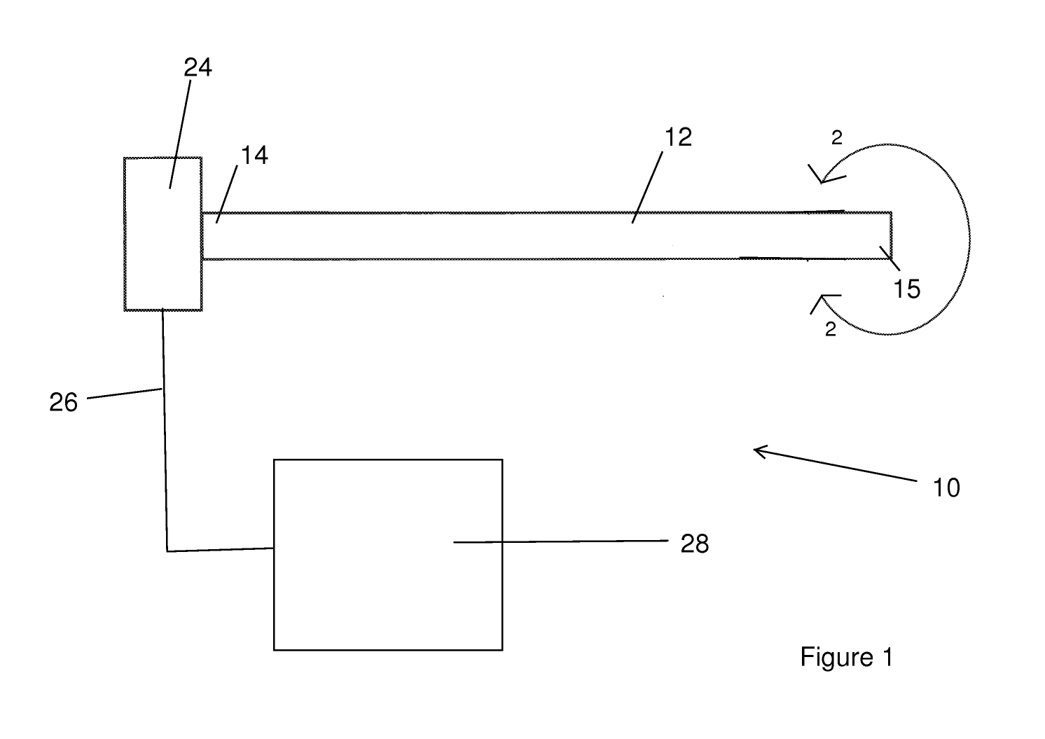

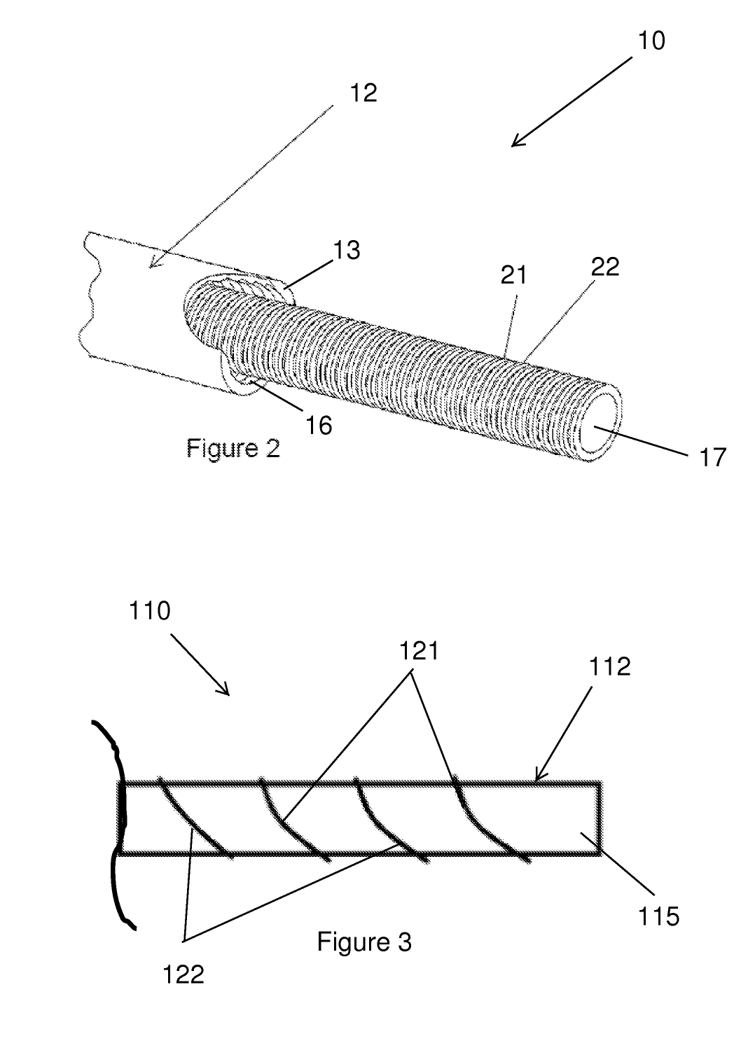

Looking at FIGS. 1 and 2, there is shown a non-limiting example embodiment of an electroporation system 10 that is useful in treating thrombosis in a subject. The term “subject” means a mammal, preferably a human. Electroporation is the phenomenon that makes cell membranes permeable by exposing them to certain electric pulses. The term “irreversible electroporation” encompasses the permeabilization of the cell membrane through the application of electrical pulses across the cell. In irreversible electroporation, the permeabilization of the cell membrane does not cease after the application of the pulse and the cell membrane permeability does not revert to normal. The cell does not survive irreversible electroporation and cell death is caused by the disruption of the cell membrane and not merely by internal perturbation of cellular components. Openings in the cell membrane are created and/or expanded in size resulting in a fatal disruption in the normal controlled flow of material across the cell membrane. The cell membrane is highly specialized in its ability to regulate what leaves and enters the cell. Irreversible electroporation destroys that ability to regulate in a manner such that the cell can not compensate and the cell dies.

The electroporation system 10 of FIGS. 1 and 2 includes a catheter body 12 having a tubular outer wall 13 extending from a proximal end 14 to a distal end 15. The body 12 defines a lumen 16. A wire 17 is positioned in the lumen 16. In one non-limiting form, the wire has a diameter of 0.5 to 3 millimeters (0.020-0.120 inches). The wire 17 may comprise a metallic material, such as stainless steel, coated in an insulating polymeric material, such as polytetrafluoroethylene. A pair of a first round wire electrode 21 and a second round wire electrode 22 are helically wound in spaced parallel relationship around the wire 17. The shape of the electrodes 21, 22 may be other than circular (e.g., oval, square, rectangular, or polygonal). The pair of electrodes 21, 22 may be wound along either the entire length of the wire, or only in a treatment zone having a longitudinal length that extends from a distal end of the catheter body 12. In one non-limiting form, the longitudinal length of the treatment zone may be between about 1 to about 10 centimeters. The electrodes 21, 22 form an anode and a cathode, respectively.

The catheter body 12 is connected at the proximal end 14 to a fitting 24 where the electrode 21 and the electrode 22 are placed in electrical communication via electrical line 26 with a voltage pulse generator 28. One non-limiting example voltage pulse generator is the ECM® 830 square wave pulse generator available from BTX Instrument Division of Harvard Apparatus, Inc., Holliston, Mass., USA.

Upon diagnosis of deep venous thrombosis, a subject is referred to the interventionalist. Either by cross-sectional imaging such as CT or MRI or by angiogram, the location and length of the deep venous thrombosis is determined. In the angiography suite, the common femoral vein is accessed. The catheter body 12 of the electroporation system 10 is inserted into the vein, and the electrode 21 and the electrode 22 are positioned adjacent a site of the thrombus. The pulse generator 28 is activated creating an electric potential between the electrodes 21, 22. The electric potential is sufficient to induce irreversible electroporation of cells of the thrombus and likely the vessel wall. The vessel wall includes three layers: an inner, middle, and outer layer that are called, respectively, the tunica intima, the tunica media, and the tunica adventitia. The electric potential can be sufficient to induce irreversible electroporation of cells of the thrombus and the vessel wall including the endothelium of the intima layer and smooth muscle cells of the media layer. Preferably, the electric potential is sufficient to induce thermal damage to vein tissue adjacent the thrombus. The non-thermal irreversible electroporation is preferably delivered along the entire length of the thrombus. The procedure may be anywhere between 30 minutes to 1 hour. The subject is then discharged with an anticoagulation therapy plan (e.g., lovenox or warfarin) and an appointment for an ultrasound study to evaluate for dissolution of the thrombus.

Electroporation protocols involve the generation of electrical fields in tissue and are affected by the Joule heating of the electrical pulses. When designing the thrombus electroporation protocol, it is important to determine the appropriate electrical parameters that will maximize tissue permeabilization without inducing deleterious thermal effects. Typical values for the pulse length for irreversible electroporation of the thrombus are in a range of from about 10 microseconds to about 10 seconds, or about 25 microseconds to about 5 seconds, or about 50 microseconds to about 100 microseconds. The pulse may be at a voltage of about 5 volts to 3,000 volts, or about 10 volts to 1000 volts, or about 50 volts to 500 volts, or about 100 volts to 200 volts for irreversible electroporation. The number of pulses may be between 1 and 200, or between 50 and 150. The size, shape and distances of the electrodes can vary and such can change the voltage and pulse duration used.

Those skilled in the art will adjust the parameters in accordance with this disclosure to obtain the desired degree of electroporation of the thrombus and avoid thermal damage to surrounding cells.

FIG. 3 is a detailed partial side view of a catheter body 112 of another electroporation system 110 that is useful in treating thrombosis in a subject. The materials used to fabricate the catheter body 112 are any suitable polymeric materials, such as thermoplastic polyurethanes, nylons, polyether block amides, ethylene vinyl acetate, silicones, polyolefin elastomers, styrenic elastomers, and polyester elastomers. A pair of a first round wire electrode 121 and a second round wire electrode 122 are helically wound in spaced parallel relationship around the catheter body 112. The shape of the electrodes 121, 122 may be other than circular (e.g., oval, square, rectangular, or polygonal). The pair of electrodes 121, 122 may be wound along either the entire length of the catheter body 112, or only in a treatment zone having a longitudinal length that extends from a distal end 115 of the catheter body 112. In one non-limiting form, the longitudinal length of the treatment zone may be between about 1 to about 10 centimeters. The electrodes 121, 122 form an anode and a cathode, respectively. The catheter body 112 may have a size of 3 to 8 on the French catheter scale. The catheter body 112 is connected at the proximal end to a fitting (similar to 24 in FIG. 1) where the electrode 121 and the electrode 122 are placed in electrical communication with a voltage pulse generator (similar to 28 in FIG. 1). The electroporation system 110 may be used to induce irreversible electroporation of cells of the thrombus as in the electroporation system 10 of FIGS. 1 and 2.

The electroporation systems 10, 110 of FIGS. 1, 2 and 3 may be used in other applications. For example, an imaging method may include the steps of positioning the pair of electrodes 21, 22 adjacent a site of a thrombus in a subject;

creating an electric potential between the pair of electrodes 21, 22 wherein the electric potential is sufficient to induce irreversible electroporation of cells of the thrombus; and acquiring an image of the site of the thrombus in the subject. In another non-limiting example, the electroporation system 10, 110 of FIGS. 1, 2 and 3 may be used in a method for promoting weight loss in a subject. The method includes the steps of inducing irreversible electroporation of cells lining a stomach in the subject. The pair of electrodes 21, 22 is positioned adjacent cells lining the stomach, and an electric potential is created between the pair of electrodes 21, 22 wherein the electric potential is sufficient to induce irreversible electroporation of cells lining the stomach.

### II. Irreversible Electroporation in the Sterility of Intravenous/Intraperiotoneal Catheters

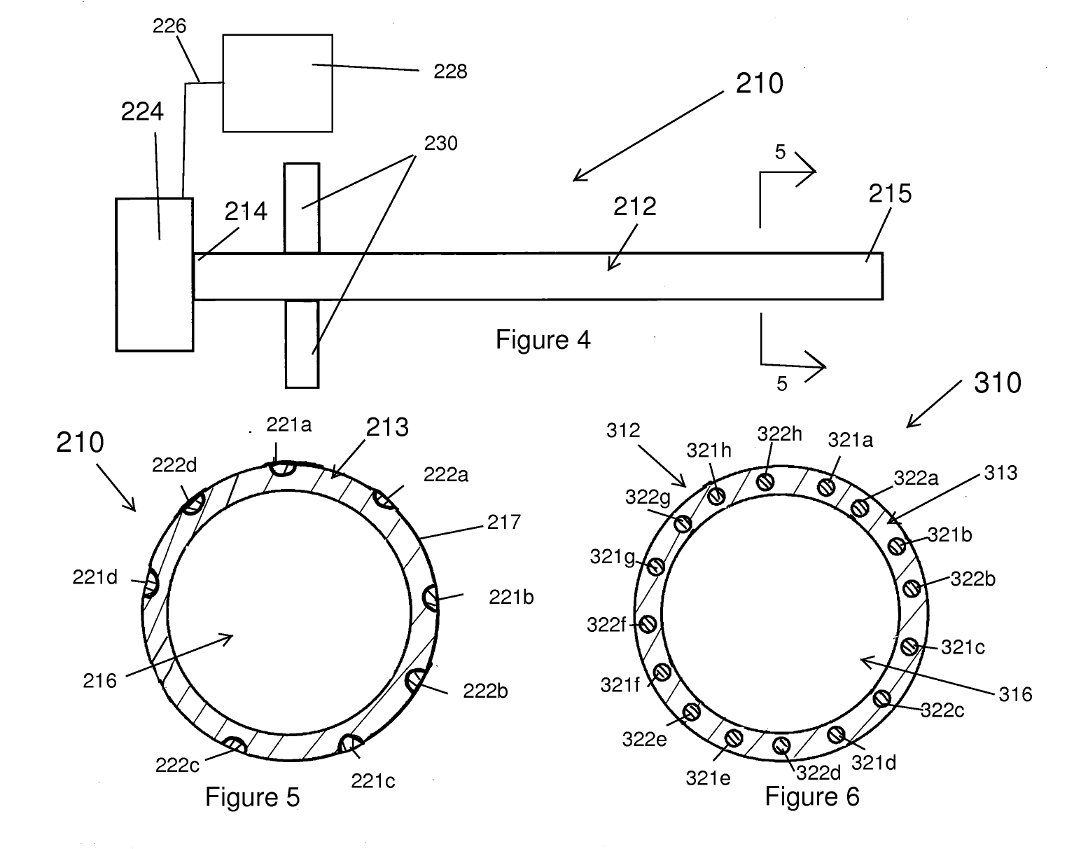

Turning now to FIGS. 4 and 5, there is shown a catheter system 210 of the present disclosure that includes means for sterilizing the catheter. The catheter system 210 includes a catheter body 212 having a tubular outer wall 213 extending from a proximal end 214 to a distal end 215. The body 212 defines a lumen 216. The materials used to fabricate the catheter body 212 are any suitable polymeric materials, such as thermoplastic polyurethanes, nylons, polyether block amides, ethylene vinyl acetate, silicones, polyolefin elastomers, styrenic elastomers, and polyester elastomers.

A first pair of a half round wire electrode 221 a and a half round wire electrode 222a run longitudinally along the outside surface 217 of the outer wall 213. Likewise, a second pair of half round wire electrodes 221b, 222b, a third pair of half round wire electrodes 221c, 222c, and a fourth pair of half round wire electrodes 221d, 222d run longitudinally along the outside surface 217 of the outer wall 213 (see FIG. 5). The pair of electrodes may be randomly spaced around the outer wall 213, or equally spaced around the outer wall 213. The four electrode pairs each form an anode and a cathode.

The catheter body 212 is connected at the proximal end 214 to a fitting 224 where the four anode, cathode pairs are placed in electrical communication via line 226 with a voltage pulse generator 228. One non-limiting example voltage pulse generator is the ECM® 830 square wave pulse generator available from the BTX Instrument Division of Harvard Apparatus, Inc., Holliston, Mass., USA. Attachment structures 230 allow fastening of the catheter body 212 to a subject's body part (e.g., skin). The attachment structures 230 may also define an indwelling portion of the outer wall 213 that lies between the attachment structures 230 and the distal end 215 of the body 213.

The four pairs of electrodes may run from the proximal end 214 to the distal end 215 of the body 213. Alternatively, the four pairs of electrodes may only run along the body 212 in a treatment zone having a longitudinal length equal to the indwelling portion of the outer wall 213. As shown in FIG. 5, each of the electrodes 221a, 221b, 221c, 221d, 222a, 222b, 222c, 222d includes a first surface in contact with the outer wall 213 of the body 212 and a second surface that is not in contact with the outer wall 213 thereby exposing a section of each of the electrodes 221a, 221b, 221c, 221d, 222a, 222b, 222c, 222d. This may increase the electroporation of cells on or adjacent the outside surface 217 of the outer wall 213.

FIG. 6 shows another catheter body 312 of another catheter system 310 of the present disclosure that includes means for sterilizing the catheter. The catheter system 310 includes a catheter body 312 having a tubular outer wall 313 extending from a proximal end to a distal end of the catheter body 312 (as in the embodiment of FIG. 4). The body 312 defines a lumen 316. The materials used to fabricate the catheter body 312 are any suitable polymeric materials, such as thermoplastic polyurethanes, nylons, polyether block amides, ethylene vinyl acetate, silicones, polyolefin elastomers, styrenic elastomers, and polyester elastomers.

Eight pairs of round wire electrodes 321a & 322a, 321b & 322b, 321c & 322c, 321d & 322d, 321e & 322e, 321f & 322f, 321g & 322g, and 321h & 322h, run longitudinally inside the outer wall 313 of the catheter body 312. The pair of electrodes may be randomly spaced inside the outer wall 313, or equally spaced inside the outer wall 313. The eight electrode pairs each form an anode and a cathode. The eight pairs of electrodes may run from the proximal end to the distal end of the body 313. Alternatively, the eight pairs of electrodes may only run inside the body 312 in a treatment zone having a longitudinal length equal to the indwelling portion of the outer wall 313. The eight anode, cathode pairs are placed in electrical communication with a voltage pulse generator (as in the embodiment of FIG. 4).

The catheter body 212 of the embodiment of FIGS. 4 and 5 or the catheter body 312 of FIG. 6 can be inserted into a body cavity, duct, or vessel. Functionally, the catheter allows drainage, administration of fluids or gases, or access by medical instruments as is known in the art. The catheter body 212 or the catheter body 312 may be part of a catheter such as, without limitation, a central venous catheter, a peripherally-inserted central catheter, a dialysis catheter, a percutaneous nephrostomy catheter, a gastrostomy tube, a urinary catheter, or a biliary drainage catheter. The catheter may be a long term catheter, that is, the catheter stays in the body for more than twenty-eight days.

When the catheter body 212 of the embodiment of FIGS. 4 and 5 or the catheter body 312 of FIG. 6 is inserted into a body cavity, duct, or vessel, an infectious agent (e.g., fungi, micro-organisms, parasites, viral pathogens, or bacterial pathogens) may over time come into contact with or become situated adjacent the outer wall 213 or 313 of the catheter body 212 or 312. The infectious agent may comprise microorganisms capable of growing a biofilm on the outer wall of the body. The pulse generator is activated creating an electric potential between each of the pairs of electrodes. The pulse length and pulse voltage and the number of pulses is selected to create an electric potential that is sufficient to induce irreversible electroporation of an infectious agent in contact with or situated adjacent the outer wall 213 or 313 of the catheter body 212 or 312. Preferably, the electric potential is insufficient to induce thermal damage to tissue adjacent the indwelling portion of the catheter body. The irreversible electroporation kills the infectious agent which may be trapped in biofilm. A treatment program may be created for the catheter body 212 or 312. For example, the pulse generator may be activated after a predetermined time period, such as every day or every week.

The invention is further illustrated in the following Examples which are presented for purposes of illustration and not of limitation.

### EXAMPLES

### Example 1

Study Design: The goal of this study was to determine whether the application of irreversible electroporation (IRE) on rat venous thrombi to ablate the cells of the venous wall and the blood clot can prevent clot organization. The ability of IRE to induce non-thermal cell death and target the cellular membrane is the primary motivation behind applying this technology. Because of the relative specificity for the cell membrane, IRE has been shown in animal studies to spare tissue scaffolds, in contrast to other modalities that cause thermal ablation. For example, experimental studies examining histologic changes after IRE performed on rat carotid arteries showed that at 4 weeks post-ablation, the vascular connective tissue matrix remained intact. The number of arterial wall vascular smooth muscle cells was decreased, but no thrombosis, necrosis, or aneurysm was present. Studies of IRE ablation in pig liver demonstrated that complete ablation of tissue adjacent to major blood vessels may be accomplished without compromising the structural integrity or functionality of blood vessels, bile ducts, and connective tissue.

Methods: A rat femoral vein ligation model was developed to produce a reproducible venous thrombus. Briefly, the common femoral vein at the level of the inguinal ligament, the proximal superficial femoral vein, muscular branch and the inferior epigastric vein were all ligated leaving the profunda femoris vein patent. Approximately 30 minutes after a venous thrombus is established, the IRE device is brought in close proximity to the venous wall (approximately 1 mm apart) and electrical pulses are delivered: an electrode with a surface area of 38 mm²was externally applied to incorporate the ligated vein segment keeping a 1 mm distance between the contact surfaces. Short square pulses were delivered using BTX 830 pulse generator (Harvard Apparatus Inc, Holliston Mass.). Specific parameters were: 80 Vmm⁻¹ field, 70 μs duration, 99 pulses, 1 Hz. In vivo currents were measured using PicoScope 4224 Oscilloscope with Pico Current Clamp (60A AC/DC) and analyzed with Pico Scope 6 software (Pico technologies Inc., UK); mean currents measured were 1.34±0.32 Amp. After NT-PEF treatment, the electrode was removed and the wound was closed using 5-0 prolene sutures and the rats were allowed to survive 3 or 7 days. At each endpoint, venous tissue segment between the two-ligation sites was harvested for histological or molecular analysis.

The contralateral femoral vein was also ligated in a similar fashion but was not treated with IRE; the untreated, negative control group. The groin skin bilaterally was then sutured and the rats were allowed to live until day 3 or day 7. Rats were anesthetized and the ligated femoral veins were removed for histologic or molecular evaluation. The IRE parameters were optimized to ensure that structural integrity of the venous wall was preserved.

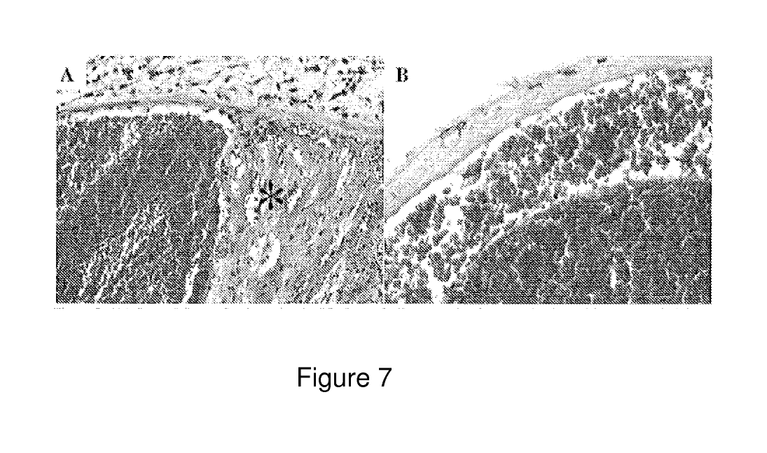

Venous thrombosis was reproducibly demonstrated in the rat femoral ligation model. At day 3 (n=7; 14 veins), application of IRE to a thrombosed femoral vein successfully preserved the scaffold of the venous wall, successfully ablated majority of the cells within the venous wall and within the thrombus and, most importantly, prevented the early stages of clot organization. See FIG. 7, which shows in (A), a control femoral vein at day 3 wherein the black star indicates early clot organization with regions of high cellularity, and in (B), IRE treated femoral vein showing preservation of the venous scaffold, minimal remaining cells post-ablation and absence of any signs of clot organization. At day 7 (n=7; 14 veins), drastic differences were noted on histology when comparing IRE treated veins to the control group of veins. IRE treated veins demonstrated a significant improvement in the patency of the vein (Lumen patency: Control 7.4%±8.1; Treated 80.4%±9.4; P<0.0001). The non-treated, control veins revealed only 3.6% (SD 2%) of the venous lumen to be patent. Additionally, IRE treated samples demonstrated minimal if any detection of collagen levels by immunohistochemistry compared to the control samples.

### Example 2

In this Example, it is shown that non-thermal, low-voltage, pulsed electric fields applied to rat deep vein thrombus prevents organization and maintains patency of the vein. In the treated veins, there are significantly less nucleated cells, and decreased levels of collagen and extracellular traps. Organization of the thrombus in the control samples was associated with increased levels of transforming growth-beta. The results suggest that this treatment as an adjunct to anticoagulation therapy may potentially increase the clearance of venous thrombi, decrease the incidence of post-thrombotic syndrome and decrease morbidity and mortality associated with deep vein thrombosis.

Deep vein thrombosis (DVT) is among the most prevalent medical problems today. Once diagnosed, it is treated by anticoagulation therapy. However, despite best medical therapy, residual thrombi are common and can lead to the development of post-thrombotic syndrome (PTS), which has an incidence of 60% in DVT patients. PTS is associated with significant morbidity and mortality; it increases the risk for DVT recurrence necessitating life-long anticoagulation. PTS also severely impacts the quality of life causing chronic venous insufficiency and, at end stage, venous ulcers. To prevent PTS, the venous thrombus must be completely dissolved; however, heterogeneity of the thrombus matrix when medical therapy is initiated, prevents its resolution leading to variable responses to anticoagulation therapy and the development of PTS. An organized thrombus matrix is resistant to endogenous thrombolysis whereas a fresh, acute thrombus is sensitive. As a thrombus ages, it undergoes active remodeling by transforming a cell-rich thrombus into a connective tissue-rich thrombus; by 21 days, up to 80% of thrombus is comprised of connective tissue such as collagen. These changes reduce the efficacy of anticoagulation therapy. In this example, it was tested whether the delivery of non-thermal, low-voltage, pulsed electric fields (NT-PEF) applied to the thrombosed vein could selectively ablate the living cells and prevent the transformation of the thrombus into lysis-resistant, collagen laden organized thrombus. This strategy has the potential for translation into the clinic as an adjunct to anticoagulation therapy to decrease the incidence of PTS.

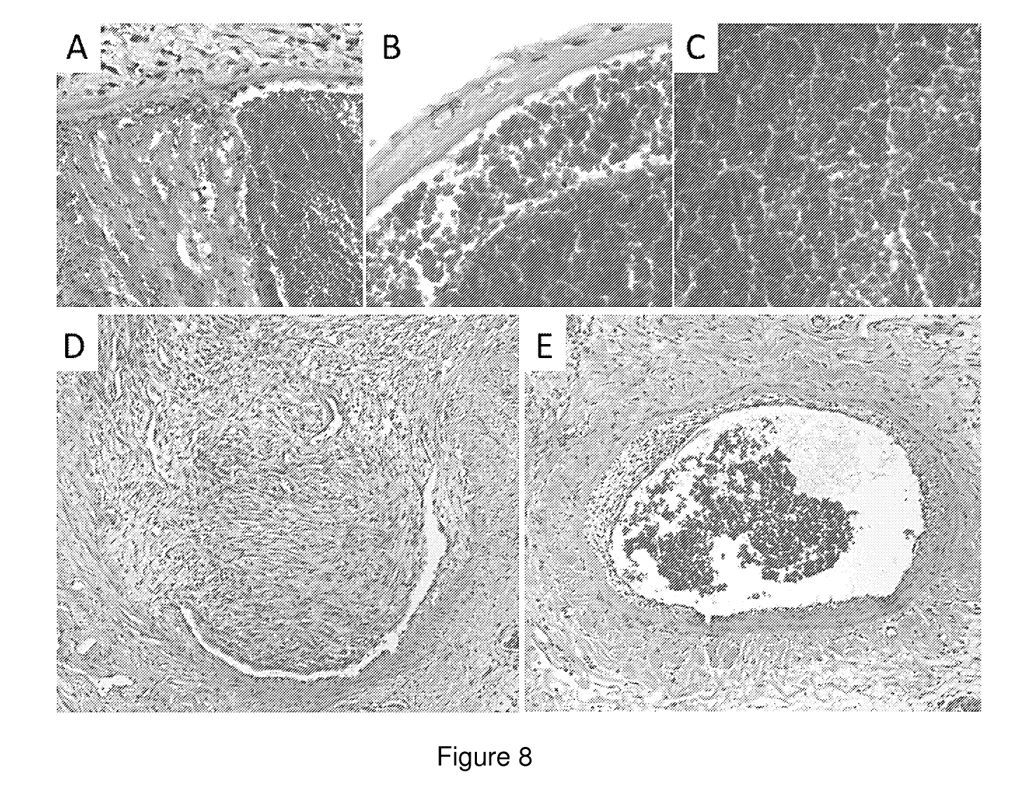

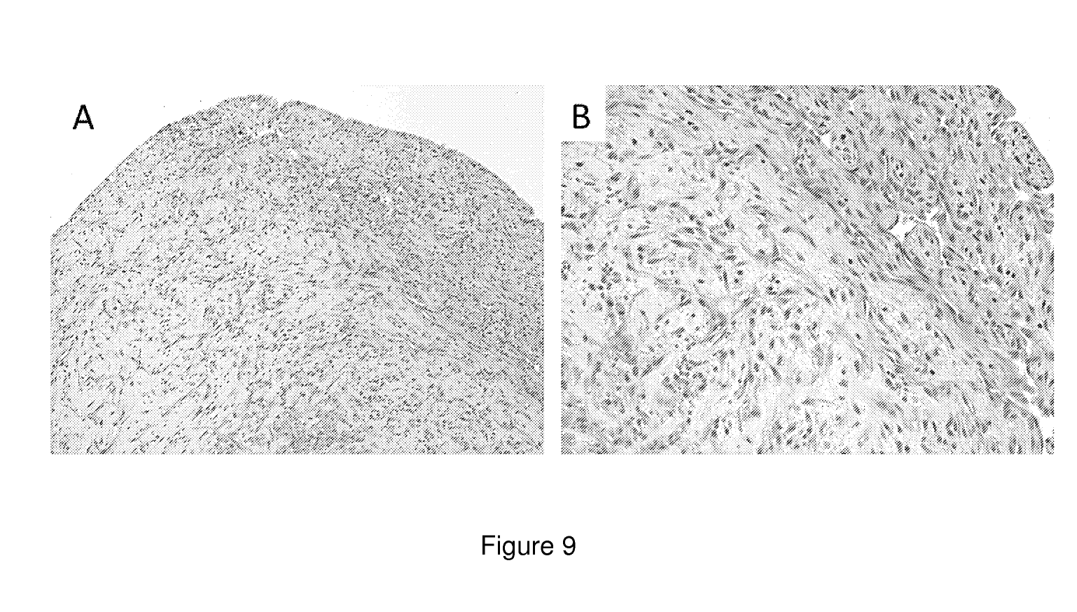

One aim was to deliver the lowest electric field possible that would decellularize the venous bed including the DVT leaving the venous matrix intact without thermal injury. By decellularizing the DVT, it was hypothesized that this would maintain the acute, degradable state of thrombus and prevent organization. To test this, a bilateral femoral vein ligation model in the rat was used to produce a DVT. Following the creation of the model, the NT-PEF device was placed in close proximity to the thrombosed vein to deliver electric pulses (N=21; Mean current 1.34±0.32 Amp; N=5). The contralateral femoral vein DVT in the same rat did not receive the electric field treatment comprising the control group (N=21). At day 3, the veins of the control group (N=7) were firm on gross inspection and the thrombus matrix on histology revealed evidence of scattered fibrin focally attached to the vessel wall with associated high cell density (FIG. 8A). At day 7, the veins of the control group (N=7) showed replacement of the fibrin rich matrix by connective tissue containing spindle shaped fibroblast cells and inflammatory cells with significant loss of vessel wall architecture (FIG. 8D). The histologic appearance of the DVT from day 3 to day 7 demonstrated marked organization of the thrombus producing a microscopic appearance similar to human chronic thrombi (FIG. 8D and 9).

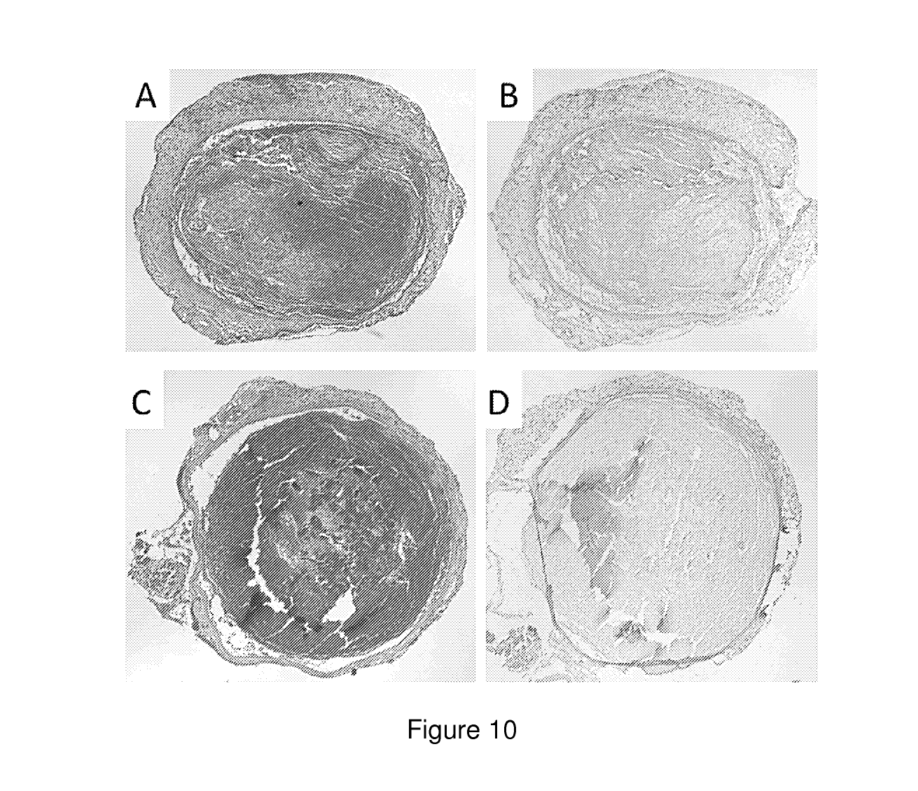

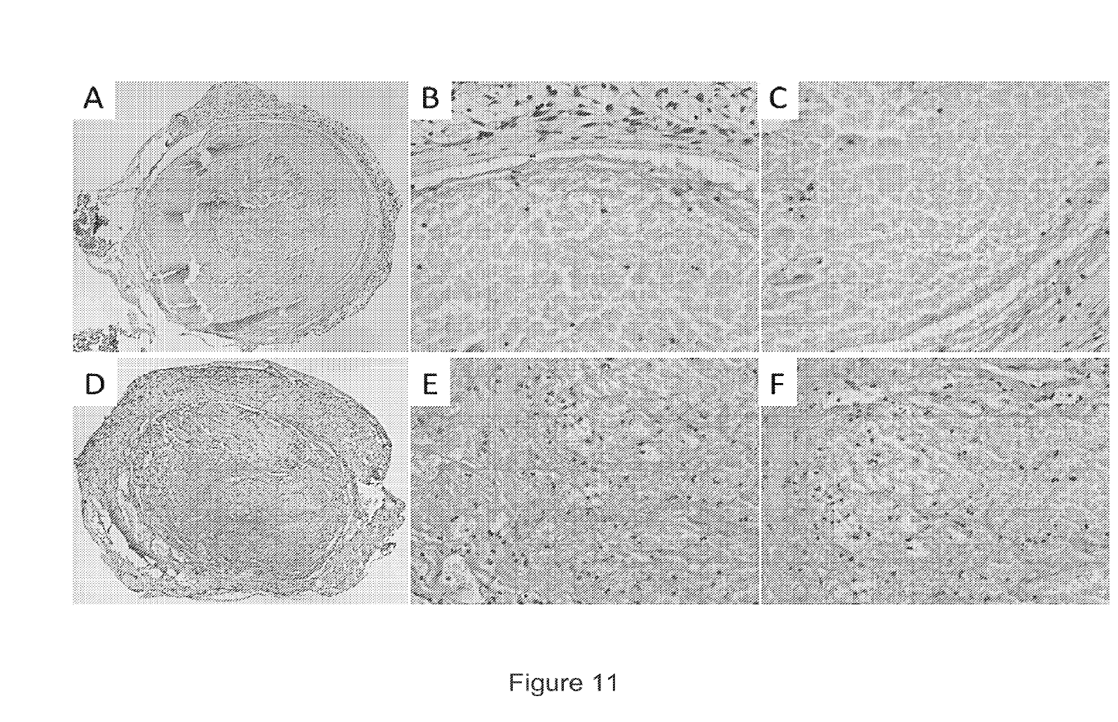

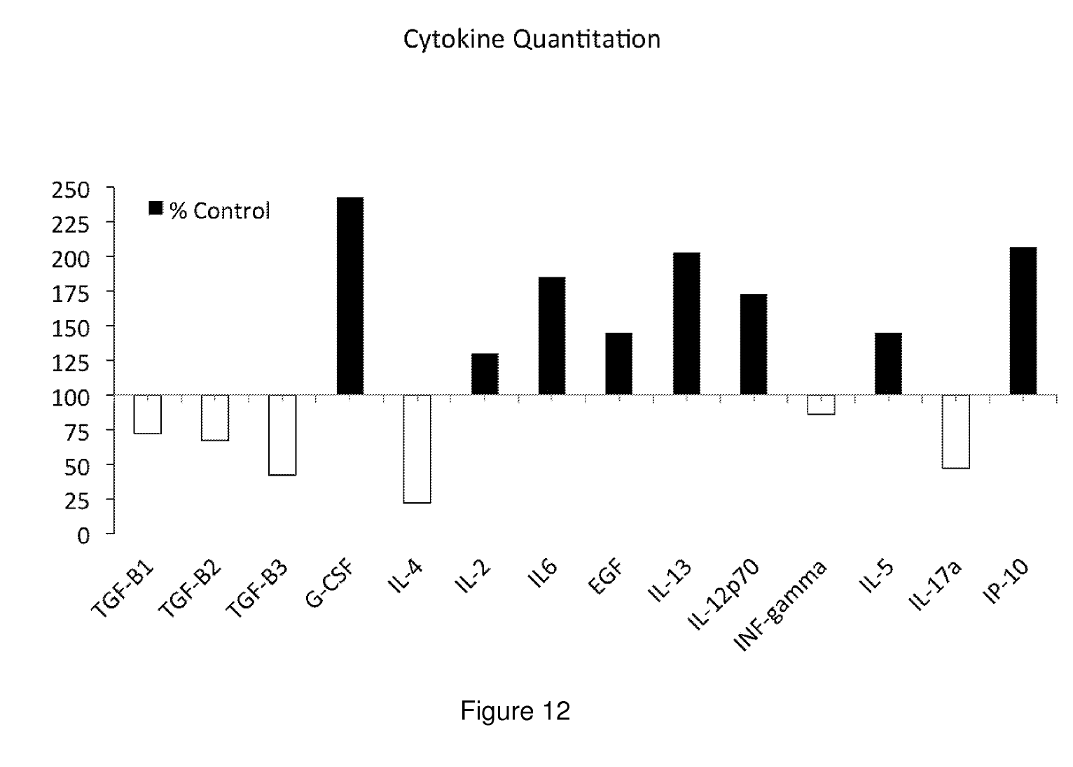

In contrast to the control group at day 3, the treated group (N=7) demonstrated veins that were soft and on occasion collapsed; on histology, there was minimal evidence of a fibrin-rich thrombus matrix and no attachments were seen to the vessel wall (FIG. 8B and 8C). The lumen contained fresh blood with minimal evidence of fibrin often along the periphery at the interface between the intima and the lumen without evidence for cellular infiltration from the vessel wall. To demonstrate that treatment led to decreased collagen deposition within the thrombus matrix, samples were immunostained for collagen-3, which is the type secreted by young fibroblasts (see FIG. 10). Consistent with histologic and gross evaluation, there was significantly decreased levels of collagen-3 detected in the treated veins compared to the control samples (treated group 24 AU±13; control group 76 AU±5; P=0.003). At day 3, treated veins demonstrated significantly decreased number of cells within the thrombus when compared to the control samples (treated group 27±8; control group 264±62; P<0.0001). These numbers were similar when MPO positive cells were counted in the two groups suggesting that the cells detected within the thrombus were neutrophils and macrophages. Consistent with decreased levels of MPO positive cells in the treated group, there was also significantly decreased prothrombogenic extracellular traps detected in the thrombus matrix (Control 77 AU±7.5; Treated 22±2.7; P<0.0001; see FIG. 11). The extracellular matrix of the venous wall was similar in both groups implying that there was no evidence of thermal injury in the treated veins. Additionally, the circumference of the treated veins compared to the control veins was similar suggesting that decellularization of the venous wall did not lead to aneurysm formation (treated group 100 AU±23; control group 107 AU±29; P=0.18). Then the concentration of 23 cytokines and transforming growth factors (TGF)-B1, B2, and B3 in tissue lysates of day 3 control group (N=7) and treated group (N=7) were quantified. Of the 23 cytokines and TGF-B proteins, 11 of the cytokines and all three of the TGF-B growth factors were significantly different between the two groups (see FIG. 12). Most notable were the TGF-B growth factors, which are key players in the process of fibrosis, were significantly decreased in the treated group (TGF-B1: Control 2509±419 pg/ml; Treated 1824±288 pg/ml; P=0.017; TGF-B2: Control 147±20 pg/ml; Treated 99±9 pg/ml; P=0.027; TGF-B3: Control 21.5±4 pg/ml; Treated 9±0.6 pg/ml; P=0.04).

At day 7, the veins of the control group (N=7) showed significant organization of the thrombus with near occlusion of the lumen and nearly complete loss of vessel wall-lumen interface (see FIG. 8D). In the treated group, however, the lumen was widely patent (see FIG. 8E) suggesting that ablation of the cells of the venous wall and the DVT using NT-PEF, organization of the thrombus was avoided (Lumen patency: Control 7.4%±8.1; Treated 80.4%±9.4; P<0.0001). The circumference of the venous wall was similar and no evidence of aneurysms was detected (Control 141 AU±26; Treated 153 AU±23; P=0.2).

To-date, the best treatment option for DVT is anticoagulation therapy, which has been shown to protect against embolism and clot extension, but does not address the detrimental effects of fibrosis within the vein lumen that are associated with clot remodeling and PTS. Translation of the technology of the present disclosure to a catheter directed intra-thrombus NT-PEF delivery as an adjunct to anticoagulation therapy has the potential to significantly decrease the morbidity and mortality associated with DVT.

### Methods

All experimental animal procedures were approved by the Institutional Animal Care and Use Committee (IACUC) and conform to the Guide for the Care and Use of Laboratory Animals published by the U.S. National Institutes of Health (NIH Publication No. 85-23, revised 1996. Male Sprague drawly rats (300-350 grams) were acquired from Charles River Laboratories (Boston, Mass., USA). Rats were anesthetized with 2% isoflurane in 100% oxygen delivered by a nose cone and placed in the supine position on a warming platform to maintain a core temperature of 37° C. using the ATC1000 system (World Precision Instruments, Sarasota, Fla., USA) with rectal probe. The groin was shaved and disinfected and a 2 cm transverse incision was made and the skin was retracted with stays and hooks. The femoral vessel bundle was exposed under surgical microscope and the vein was separated from the nerve and artery using a combination of sharp and blunt dissection. To induce venous thrombosis, the femoral vein segment was ligated using a 5-0 silk ligatures placed below the infrainguinal ligament and distal to the inferior epigastric branch. All visible side branches including the inferior epigastric and the muscular branch were ligated except for the profunda femoris vein. The surgery was repeated on the contra lateral hind limb in the same animal. One side was subjected to NT-PEF treatment while the other side served as control.

### Non-Thermal Irreversible Electroporation

Following femoral vein ligation, an electrode with a surface area of 38 mm² was externally applied to incorporate the ligated vein segment keeping a 1 mm distance between the contact surfaces. Short square pulses were delivered using BTX 830 pulse generator (Harvard Apparatus Inc., Holliston Mass., USA). NT-PEF specific parameters were: 80 Vmm⁻¹ field, 70 ps duration, 99 pulses, 1 Hz). In vivo currents were measured using PicoScope 4224 Oscilloscope with Pico Current Clamp (60A AC/DC) and analyzed with Pico Scope 6 software (Pico Technologies Inc., United Kingdom). After NT-PEF treatment, the electrode was removed and the wound was closed using 5-0 prolene sutures and the rats were allowed to survive 3 or 7 days. At each endpoint, venous tissue segment between the two-ligation sites was harvested for histological or molecular analysis.

Thus, the invention provides methods and devices for treating conditions of tubular body structures, such as veins.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A method for treating thrombosis in a subject, the method comprising:
(a) inducing irreversible electroporation of cells of a thrombus and cells of a vessel wall in the subject.

2. The method of claim 1 wherein:
step (a) comprises: (i) positioning a pair of electrodes adjacent a site of the thrombus or within the thrombus, (ii) creating an electric potential between the pair of electrodes, the electric potential being sufficient to induce irreversible electroporation of cells of the thrombus and the cells of the vessel wall including the endothelium of the intima layer and smooth muscle cells of the media layer.

3. The method of claim 2 wherein:
the electric potential is sufficient to induce thermal damage to vein tissue adjacent the thrombus.

4. The method of claim 2 wherein:
the electric potential is created by a voltage pulse generator in electrical communication with the pair of electrodes.

5. The method of claim 2 wherein:
the pair of electrodes are wound in spaced parallel relationship around a wire.

6. The method of claim 3 wherein:
the wire has a diameter of 0.5 to 3 millimeters.

7. The method of claim 3 wherein:
the pair of electrodes are positioned in a treatment zone having a longitudinal length that extends from a distal end of the wire.

8. The method of claim 7 wherein:
the longitudinal length is between 1 to 10 centimeters.

9. The method of claim 2 wherein:
the pair of electrodes are wound in spaced parallel relationship around a catheter.

10. The method of claim 9 wherein:
the catheter has a size of 3 to 8 on a French catheter scale.

11. The method of claim 9 wherein:
the pair of electrodes are positioned in a treatment zone having a longitudinal length that extends from a distal end of the catheter.

12. The method of claim 11 wherein:
the longitudinal length is between 1 to 10 centimeters.

13. The method of claim 2 wherein:
the pair of electrodes span an entire length of the thrombus.

14. The method of claim 1 wherein:
the method inhibits collagen deposition within the thrombus.

15. The method of claim 1 wherein:
the thrombosis is a venous thrombosis.

16. The method of claim 1 wherein:
the thrombosis is a deep vein thrombosis.

17. The method of claim 1 further comprising:
(b) administering an anticoagulant to the subject.

18. The method of claim 1 wherein:
the thrombus is less than two weeks old.

19. The method of claim 1 wherein:
the thrombus has less than 80% collagen, preferably less than 70% collagen, preferably less than 60% collagen, preferably less than 50% collagen, preferably less than 40% collagen, preferably less than 30% collagen, preferably less than 20% collagen, preferably less than 10% collagen.

20. An imaging method comprising:
(a) positioning a pair of electrodes adjacent a site of a thrombus in a subject;
(b) creating an electric potential between the pair of electrodes, the electric potential being sufficient to induce irreversible electroporation of cells of the thrombus; and
(c) acquiring an image of the site of the thrombus in the subject.

21. A method for promoting weight loss in a subject, the method comprising:
(a) inducing irreversible electroporation of cells lining a stomach in the subject.

22. The method of claim 21 wherein:
step (a) comprises: (i) positioning a pair of electrodes adjacent cells lining the stomach, (ii) creating an electric potential between the pair of electrodes, the electric potential being sufficient to induce irreversible electroporation of cells lining the stomach.

23. The method of claim 22 wherein:
the electric potential is created by a voltage pulse generator in electrical communication with the pair of electrodes.

24. The method of claim 22 wherein:
the pair of electrodes are wound in spaced parallel relationship around a wire.

25. The method of claim 24 wherein:
the wire has a diameter of 0.5 to 3 millimeters.

26. The method of claim 22 wherein:
the pair of electrodes are wound in spaced parallel relationship around a catheter.

27. A catheter system comprising:
a body having an outer wall extending from a proximal end to a distal end, the body defining at least one lumen;
a pair of electrodes disposed in the outer wall of the body;
a voltage generator in electrical communication with the pair of electrodes, the voltage generator creating an electric potential between the pair of electrodes when the voltage generator is activated, the electric potential being sufficient to induce irreversible electroporation of cells of an infectious agent in contact with or adjacent the outer wall of the body.

28. The catheter system of claim 27 wherein:
the voltage generator is a voltage pulse generator.

29. The catheter system of claim 27 further comprising:
one or more additional pairs of electrodes disposed in the outer wall of the body, each additional pair of electrodes being in electrical communication with the voltage generator.

30. The catheter system of claim 29 wherein:
the pair of electrodes and each additional pair of electrodes are equally spaced around the outer wall of the body.

31. The catheter system of claim 29 wherein:
the pair of electrodes and each additional pair of electrodes include a first surface in contact with the outer wall of the body and a second surface that is not in contact with the outer wall of the body.

32. The catheter system of claim 29 wherein:
the pair of electrodes and each additional pair of electrodes are embedded within the outer wall of the body such that the pair of electrodes and each additional pair of electrodes are surrounded by material comprising the outer wall of the body.

33. The catheter system of claim 27 wherein:
the pair of electrodes are positioned in a treatment zone of outer wall of the body, the treatment zone having a length that extends from a distal end of the body to a location spaced from the proximal end of the body.

34. The catheter system of claim 27 wherein:
the electric potential is insufficient to induce thermal damage to tissue adjacent an indwelling portion of the outer wall of the body.

35. The catheter system of claim 27 wherein:
the infectious agent comprises microorganisms capable of growing a biofilm on the outer wall of the body.

36. The catheter system of claim 35 wherein:
the microorganisms comprise bacteria.

37. The catheter system of claim 27 wherein:
the body comprises a central venous catheter.

39. The catheter system of claim 27 wherein:
the body comprises a peripherally-inserted central catheter.

40. The catheter system of claim 27 wherein:
the body comprises a dialysis catheter.

41. The catheter system of claim 27 wherein:
the body comprises a percutaneous nephrostomy catheter.

42. The catheter system of claim 27 wherein:
the body comprises a gastrostomy tube.

43. The catheter system of claim 27 wherein:
the body comprises a urinary catheter.

44. The catheter system of claim 27 wherein:
the body comprises a biliary drainage catheter.

45. A method for maintaining sterility of a catheter, the method comprising:
(a) providing a catheter including a body having an outer wall extending from a proximal end to a distal end, the body defining at least one lumen, the catheter including a pair of electrodes disposed in the outer wall of the body; and
(b) creating an electric potential between the pair of electrodes, the electric potential being sufficient to induce irreversible electroporation of cells of an infectious agent in contact with or adjacent the outer wall of the body.

46. The method of claim 45 wherein:
step (b) comprises using a voltage generator in electrical communication with the pair of electrodes to create the electric potential between the pair of electrodes.

47. The method of claim 46 wherein:
the voltage generator is a voltage pulse generator.

48. The method of claim 46 wherein:
one or more additional pairs of electrodes are disposed in the outer wall of the body, each additional pair of electrodes being in electrical communication with the voltage generator.

49. The method of claim 48 wherein:
the pair of electrodes and each additional pair of electrodes are equally spaced around the outer wall of the body.

50. The method of claim 48 wherein:
the pair of electrodes and each additional pair of electrodes include a first surface in contact with the outer wall of the body and a second surface that is not in contact with the outer wall of the body.

51. The method of claim 48 wherein:
the pair of electrodes and each additional pair of electrodes are embedded within the outer wall of the body such that the pair of electrodes and each additional pair of electrodes are surrounded by material comprising the outer wall of the body.

52. The method of claim 45 wherein:
the electric potential is insufficient to induce thermal damage to tissue adjacent an indwelling portion of the outer wall of the body.

53. The method of claim 45 wherein:
the infectious agent comprises microorganisms capable of growing a biofilm on the outer wall of the body.

54. The method of claim 45 wherein:
the microorganisms comprise bacteria.

55. The method of claim 45 further comprising:
repeating step (b) after a predetermined time period.

56. The method of claim 45 wherein:
the catheter is selected from the group consisting of central venous catheters, peripherally-inserted central catheters, dialysis catheters, percutaneous nephrostomy catheters, gastrostomy tubes, urinary catheters, and biliary drainage catheters.

57. The method of claim 45 wherein:
the catheter is a long term catheter.
